(19) United States
(12) Plant Patent Application Publication
(10) Pub. No.: US 2011/0010812 P1
(43) Pub. Date: Jan. 13, 2011

(54) Eichhornia crassipes plant named 'Johann Schoofs Sen.'

(50) ;

(75) Inventors: Maria Rogmans, Kalkar (DE); Hermann-Josef Wilhelm, Kalkar (DE)

Correspondence Address: BUCHANAN, INGERSOLL & ROONEY PC, POST OFFICE BOX 1404, ALEXANDRIA, VA, 22313-1404, US

(73) Assignee: MARIA ROGMANS, Kalkar (DE)

(21) Appl. No.: 12/801,390

(22) Filed: Jun. 7, 2010

(30) Foreign Application Priority Data
Jun. 5, 2009 (EP) 2009/0985

(51) Int. Cl. A01H 5/00

(52) U.S. Cl. PLT/342

(57) ABSTRACT

A new Eichhornia crassipes plant named ‘Johann schoofs Sen’ is provided which is well suited to serve as a biomass renewable energy source. The plant is particularly well suited to metabolize carbon dioxide from emissions. A free-floating small compact mat-like growth habit is displayed with an intertwined root ball. A typical leaf petiole is absent, and the roots are finely structured. The leaves are dark green in coloration, and the plant survives well under low-light conditions. The plant generates itself from new shoots and commonly can double its biomass in approximately 10 to 14 days.

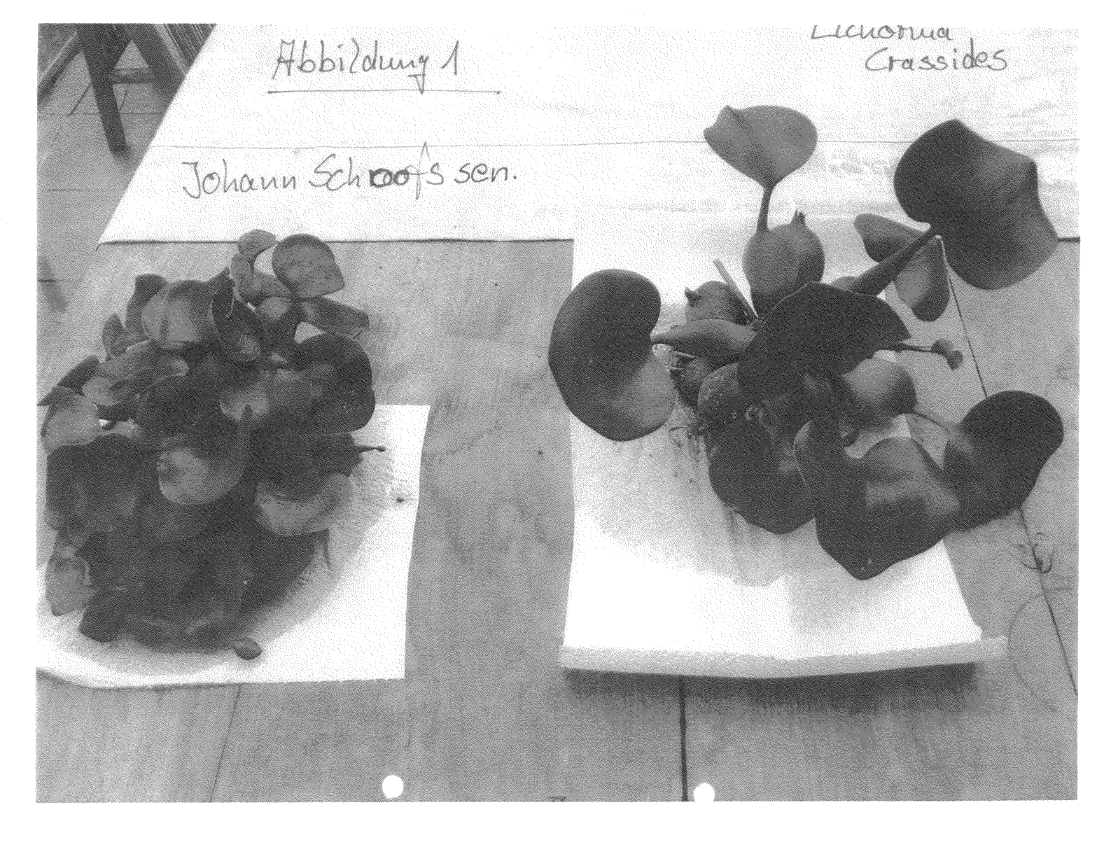

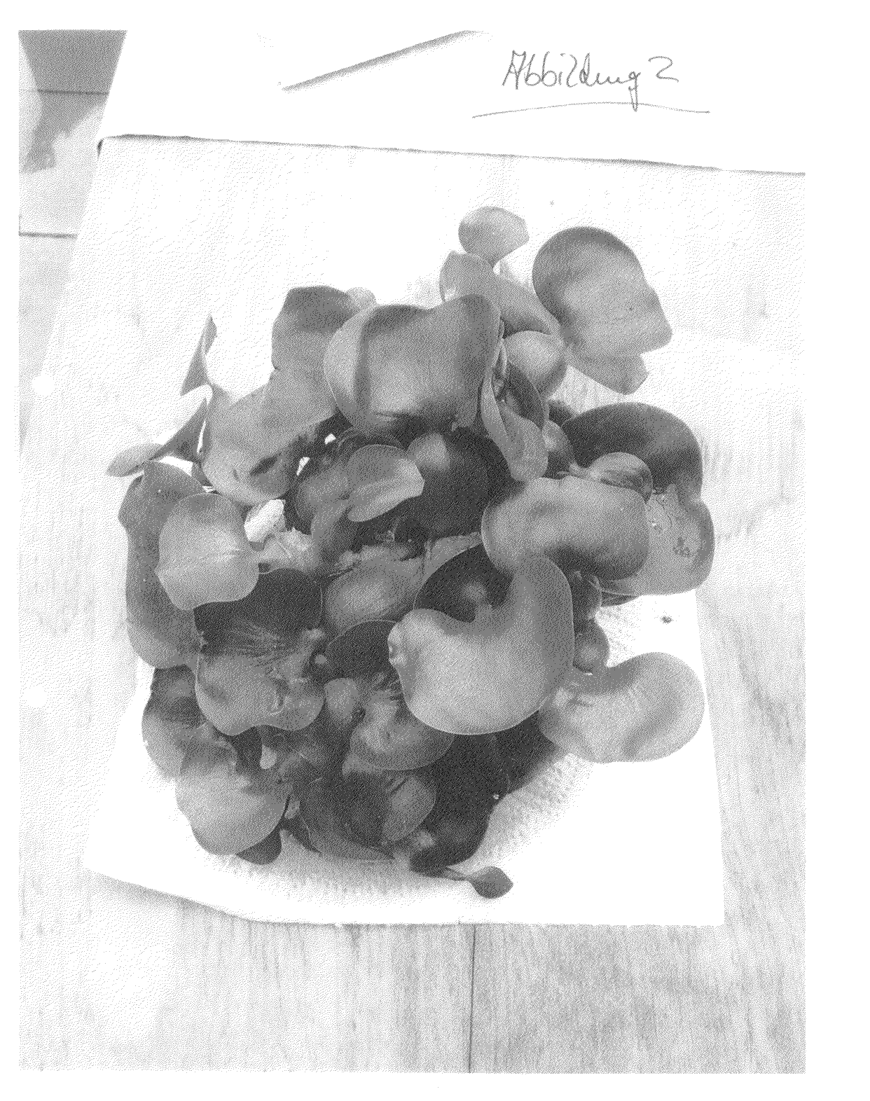

### DESCRIPTION OF VARIETY

### Botanical Taxon

### GENUS

EICHHORNIA

### SPECIES

EICHHORNIA CRASSIPES

### COMPARISON SPECIES

ditto

### New Variety

Variety denomination: Eichhornia Johann Schoofs Sen.

### Breeding Process

Breeding has been carried out by selection, i.e. by selective breeding, by which a large number of plants of several varieties of Eichhornia were kept in several containers for a longer period. The autonomous propagation of the plant happened via self-generated offshoots and stolons.

Afterwards, more selecting and separating was performed and the selection increased until the new variety was stable, homogeneous and absolutely reproducible in its appearance and form.

After successful selection, it was of no question any more whether the plants were kept under low light conditions or not. After successful selective breeding, these features were stable independently of the light conditions.

The objective of the breeding was to generate a robust and strong aquatic biomass-producing plant whose metabolic activity would also be good under low light conditions due to its strong green colour. These new distinctive features in the new variety led to a significant and well visible change of form in comparison with the original varieties.

### MORPHOLOGICAL DESCRIPTION

Form, size, colour:
General description:

The new variety forms a mat, which is free-floating on the water and which has an intertwined root ball. Its diameter amounts to approximately 20 to 25 cm and is thus remarkably smaller than the comparison variety. At the root, rather thickened petioles (which are no petioles any more) and whose bulge is pear-shaped, form what is referred to as bulbs. These are clearly stronger and more firmly shaped in the new variety than in the comparison variety. On each bulb grows at least one succulent leaf. A further significant and very visible feature is that in the comparison variety there is a long petiole of several centimetres between bulb and leaf. This petiole is completely missing in the new variety so that the leaf sits directly on the bulb, which gives the plant a distinctly more compact appearance than the comparison variety.

### DETAILED DESCRIPTION

Bulbs (inflated petioles): The bulbs show, due to their arrangement with the leaf, a lower side and an upper side, wherein the upper side of the leaf is attached quasi parallel, or is situated with the same orientation to the upper side of the bulb. The bulbs, as are the leaves, are of different colour on the upper and lower sides. The colour of the upper side is Green 42, RHS 137 C, and the colour of the lower side is Yellow Green 44 RHS 145 A. In this, the colour transitions between the upper and the lower side are merging. The number of bulbs per root ball is between 25 and 30 and, if applicable, more. The bulbs have an oval diameter and their measurement is from 1.5 to 1.8 cm over the short diameter and from 4 to 4.5 cm over the long diameter. The surface of the bulbs shows, both on the upper and the lower side, an onion-like line structure, which is visible and tangible.
Leaves: The leaves, which sit almost directly on top of the bulbs, are kidney-shaped and slightly waved and convex. The colour of the upper side of the leaf is Dark Green 41, RHS 137 A, Green 42, RHS 137 C. The colour slightly changes between these shades. The lower side is Dark Green 41 RHS 144 A. The significantly short petiole of the leaf is Yellow Green 44, RHS 145 A. The leaves of a plant attached to a coherent root ball are of different sizes, ranging from 3 to 9 cm on the long side of the kidney-shaped leaves and from 2.5 to 4.5 cm on the short side of the kidney-shaped leaves. The surface of the leaf has a distinctive, visible and tangible nerve structure with a leathery, silky matte, shining surface structure, succulent and firm.
Roots: The whole plant is a free-floating plant, which sits on its root ball in the water, wherein the root ball is completely covered by water. The roots have the form or a bird feather or pipe cleaner, whose structure in comparison with the comparison variety is clearly more finely structured, and they are pointed at the ends. Due to the finer structure the absorbing capacity is higher due to an increased biologically or metabolically active surface of the root, which is confirmed by the rich spectrum of ingredients the new variety boasts. This is the case also in the presence of low light, which is caused and optimized by the specific morphological changes in the new variety.

Photo 1: The plant in the right-hand part of the picture shows the comparison variety Eichhornia Crassipes. The left-hand plant is the candidate variety Johann Schoofs. In this connection, the pronounced compactness as well as the clearly shorter lengths of the petioles between leaf and bulb are remarkable.

Photo 2: Here, the candidate variety is clearly shown. The morphological features described above are clearly visible.

### DISTINCTIVE FEATURES WITH RESPECT TO THE COMPARISON VARIETY

Size: In contrast to the comparison variety, the new variety is remarkably smaller.
Roots: In contrast to the comparison variety the roots are remarkably more finely structured.
Readiness to grow: The new variety is definitely more ready to grow than the comparison variety and smaller in its whole form, but more compact.
Colour: The colours are considerably more intense than in the comparison variety and turn out according to the colour scheme described above. Otherwise colour indications as given above.
Supplemental breeding scheme: The original plants were kept in a room under low light conditions from the beginning. The original plant is a free-floating plant, which is exposed to strong light. However, selection was carried out according to atypical criteria. This means that selection exclusively focussed on the candidates that were able to survive under low light conditions. It was precisely those that showed a clearly stronger dark green colouring, particularly in the leaves. The selective breeding according to low light criteria has in the following generations shown the visible distinct change of form, more precisely, a remarkably short petiole between bulb and leaf and a stable dark colouring on the upper side of the leaf.
Metabolic activity: Due to the remarkably more compact form of the new variety it has a clearly more intense green colouring.

The new variety grows definitely better under increased CO₂ conditions. Thus the new variety is particularly suited to effectively metabolize CO₂ from emissions with a simultaneous increase of mass. Due to the spectrum of ingredients, the biomass can be used for what is referred to as cascade application, which means:
1^{st} step: binding of CO₂ as biocatalyst.
2^{nd} step: extraction of chemical ingredients.
3^{rd} step: energetic exploitation of residue.
4^{th} step: recycling of CO₂ resulting from the energetic exploitation in 1^{st} step.
Reproduction of the plant: The reproduction of the plant is vegetatively. The plant generates itself new shoots, and double its biomass each 10 to 14 days. Then the new plant parts of the shoots can be cut of and cultured and reproduced further. The plant is self reproductive. No seeds necessary. The plant is avaible by the applicants address.

1. New ECHHORNIA CRASSIPES, named EICHHORNIA CRASSIPES ‘Johann Schoofs sen’, with the described morphological features, according to EU variety JOHANN SCHOOFS SEN , CPVO 2009/0985.
